(19) United States
(12) Patent Application Publication
(10) Pub. No.: US 2006/0172015 A1
(43) Pub. Date: Aug. 3, 2006

(54) Methods and pharmaceutical compositions for inhibiting metastasis of malignant tumors and growth of leukemic cells

(75) Inventors: Rey-Yuh Wu, Xizhi City (TW); Bor-Shiun Chen, Xizhi City (TW); Shan-Wen Tang, Taipei (TW); Wen-Yi Kao, Xizhi City (TW); Peng-Nien Sui, Xizhi City (TW); Ching-Chueh Hsu, Xizhi City (TW); Jue-Hao Liu, Xizhi City (TW); Hsiu-Ying Chang, Xizhi City (TW)

Correspondence Address: LADAS & PARRY, 26 WEST 61ST STREET, NEW YORK, NY, 10023, US

(73) Assignees: DEVELOPMENT CENTER FOR BIOTECHNOLOGY; LEADER MACHINE CO., LTD.

(21) Appl. No.: 11/322,151

(22) Filed: Dec. 29, 2005

(30) Foreign Application Priority Data
Dec. 31, 2004 (TW) 093141926

(51) Int. Cl. A61K 33/24

(52) U.S. Cl. 424/649; 977/906

(57) ABSTRACT

The present invention provides methods for inhibiting metastasis of malignant tumors and growth of leukemic cells, which comprise administering a pharmaceutically effective amount of nano-gold. The present invention also provides pharmaceutical compositions, which comprise a pharmaceutically effective amount of nano-gold and are useful in inhibiting metastasis of malignant tumors and growth of leukemic cells.

### FIELD OF THE INVENTION

The present invention pertains to methods and pharmaceutical compositions for inhibiting metastasis of malignant tumors and growth of leukemic cells, which relate to the use of nano-gold.

### BACKGROUND OF THE INVENTION

Nano-technology is a newly rising technology which has been vigorously developed in recent years. In general, “nano-scaled materials” refer to materials having a particle size less than 100 nm. It is known that when a substance exists in a nano-scaled particle size, the physical properties thereof show dramatic differences as compared to its inherent properties. Kubo a Japanese physicist, revealed the “quantum restriction theory” in 1962 in order to explain the discontinuity of energy of nano-scaled metal particles. According to the theory, the decrease of atom numbers of nano-scaled metal particles causes an increase of the distance between the atoms.

Thus, the continuous energy that electrons inherently have is interrupted and the discontinuity of energy occurs, which leads to the change that the nano-scaled metal particles show dramatic differences in physical properties as compared to the properties that they inherently have.

Gold is known to be used in biomedical field. For example, it is known that gold has the functions of causing sedateness, tranquilization, and detoxification, and is effective in the treatment of seizure, palpitation, abscess, and rheumatoid arthritis.

Manufactures and applications of micro-particles of gold are seen in various published references. For example, ROC (Taiwan) Patent Publication No. 567091, published on 21 Dec. 2003, discloses a method for preparing nano-grade gold particles, which comprises (a) providing a solution of glyoxylic acid and adjusting the pH value of the glyoxylic acid solution to generate glyoxylate ion, and (b) utilizing the glyoxylate ion, which serves as a reducing agent, to reduce trivalence gold ion of the solution to form nano-grade gold particles. The nano-grade gold particles obtained therefrom may be implemented in the manufacture of single electron transistors.

US Patent Publication No. 2003/0116501, published on 26 Jun. 2003, provides a process using biological method for the preparation of immobilized nano-sized metal particles (e.g., gold), by utilizing fungi that naturally occurs in an aqueous medium. This US patent publication does not disclose any applications of the metal particles produced from the process.

Moreover, PRC Patent Publication No. CN 1467050 A (PRC Patent Application No. 02140720.7) discloses a method for preparing micro gold particles. This PRC patent publication generally describes that the micro gold particles produced from the method are effective in inhibiting malignant tumors and increasing the ability of immune system. However, as a matter of fact, the method of this PRC patent publication only produces gold particles having larger particle sizes, or golden sheets (which are commonly referred to as “golden foils”), but cannot produce gold particles having a nano-scaled particle size.

Generally, the “growth” of malignant tumors refers to the situation that malignant tumor cells undergo division and proliferation at the primary tumor site. The “metastasis” of malignant tumors refers to the situation that a portion of malignant tumor cells falls off the primary tumor site, invade blood vessels or lymphatic system to access to the cavity of human body and reach the other parts of human body (e.g., the pleura or peritoneum) or into the organs of human body, and then continue to undergo division arid proliferation at the metastasis regions or in the organs to form secondary malignant tumors.

Earlier clinical trails have discovered that the metastasis of solid malignant tumors is mainly through hematogenic metastasis. Because there are many connections between lymphatic system and blood vessels, in theory, malignant tumor cells can migrate back and forth between the lymphatic system and vascular system. However, the role of lymphangiogenesis in the metastasis of malignant tumors and the migration of fallen malignant tumor cells in lymphatic system is rarely understood. In recent years, it has been noted that lymphangiogenesis plays a role in promoting the lymphogenic metastasis of malignant tumors. Also, it has recognized that vascular endothelial cell growth factors (VEGF) C and D (i.e., VEGF-C and VEGF-D) are highly relevant to lymphangiogenesis.

Important cell surface receptors having an intrinsic tyrosine kinase activity, such as VEGFR-3, are known as receptor tyrosine kinases (RTK). Due to binding with ligands, the tyrosine kinase activity of cell surface receptors is activated, which leads to self-phosphorylation of tyrosine. Phosphorylation of tyrosine is the initiating event of various signal transduction pathways. VEGFR-C is a ligand for VEGFR-3. In adult tissues, the receptor of VEGF-C, “VEGFR-3,” is only expressed in lymphatic endothelium cells. Therefore, VEGFR-3 is considered as the major modulating factor of lymphangiogenesis. Moreover, relevant researches evidence that VEGFR-3 is in charge of the production of lymphatic endothelium cells. VEGFR-D is another ligand for VEGFR-3, which can also induce lymphangiogenesis in transgenic mice. In recent years, researches further evidence that VEGFR-3 is also expressed in several malignant tumors in human, such as lung adenocarcinoma, colorectal adenocarcinoma, and head and neck carcinoma. The inventors believe that drugs capable of blocking signal transduction pathways are effective in inhibition of lymphangiogenesis.

### SUMMARY OF THE INVENTION

The present invention provides methods for inhibiting metastasis of malignant tumors and growth of leukemic cells, which comprise administering a pharmaceutically effective amount of nano-gold.

The present invention also provides pharmaceutical compositions, which comprise a pharmaceutically effective amount of nano-gold and are useful in inhibiting metastasis of malignant tumors and growth of leukemic cells.

### BRIEF DESCRIPTION OF DRAWINGS

FIG. 1A shows the spectrum of energy dispersive X-ray (EDX) analysis of the NG-gp utilized in the present invention.

FIG. 1B shows the topically magnified spectrum of FIG. 1A.

FIG. 2A shows the electronic microscope analysis spectrum of the NG-25s utilized in the present invention, detected by transmitting electronic microscope (TEM).

FIG. 2B shows the electronic microscope analysis spectrum of the NG-40s utilized in the present invention detected by TEM.

FIG. 3 shows the number of nodules present in the liver of a BALB/c mouse after fourteen days since the mouse being subjected to intra-splenic implantation of a CT-26 colorectal adenocarcinoma cell line, and administration of NG-gp.

FIG. 4 shows the number of nodules present in the liver of a BALB/c mouse after fourteen days since the mouse being subjected to intra-splenic implantation of a CT-26 colorectal adenocarcinoma cell line, and administration of NG-25s.

FIG. 5 shows the number of nodules present in the liver of a BALB/c mouse after fourteen days since the mouse being subjected to intra-splenic implantation of a CT-26 colorectal adenocarcinoma cell line, and administration of NG-40s.

FIG. 6 shows the weight ratio of spleen to body of a BALB/c mouse after fourteen days since the mouse being subjected to intra-splenic implantation of a CT-26 colorectal adenocarcinoma cell line, and administration of NG-40s.

FIG. 7 shows the weight ratio of liver to body of a BALB/c mouse after fourteen days since the mouse being subjected to intra-splenic implantation of a CT-26 colorectal adenocarcinoma cell line, and administration of NG-40s.

FIG. 8 shows the influence of NG-40s on prolonging the lifespan of a BALB/c mouse implanted with a CT-26 colorectal adenocarcinoma cell line.

FIG. 9 shows the concentrations of various cytokines in a conditioned culture medium, where the human peripheral blood mononuclear cells (hPBMC) comprised therein are stimulated by NG-gp of various concentrations.

FIG. 10 shows the effectiveness of the conditioned culture mediums, where the human peripheral blood mononuclear cells (hPBMC) comprised therein are stimulated by NG-gp of various concentrations, in inhibiting the growth of a U937 tumor cell line.

FIG. 11 shows the analysis of activities of various tested articles in inhibiting the phosphorylation of VEGFR-3 tyrosine in vitro.

### DETAILED DESCRIPTION OF THE INVENTION

After extensive research, the inventors have discovered that nano-gold has an activity in inhibiting VEGF-C/VEGFR-3 signal transduction pathways, can effectively inhibit lymphangiogenesis, and thus is capable of inhibiting the metastasis of malignant tumors.

The inventors have further discovered that nano-gold is effective in inhibiting the growth of leukemic cells, and thus are useful in the treatment of leukemia.

Therefore, in one aspect, the present invention provides methods for inhibiting metastasis of malignant tumors and growth of leukemic cells, which comprise administering a pharmaceutically effective amount of nano-gold.

In another aspect, the present invention provides pharmaceutical compositions, which comprise a pharmaceutically effective amount of nano-gold and are useful in inhibiting metastasis of malignant tumors and growth of leukemic cells.

In general, the term “malignant tumors” used herein refers to colorectal cancer, hepatoma, brain tumor, lung cancer, prostate cancer, breast cancer, gastric cancer, esophageal cancer, bladder tumor, skin cancer, leukemia, pancreatic cancer, ovarian cancer, cervical cancer, and lymphoma. In particular, it refers to colorectal cancer.

The organs to which malignant tumors metastasize may include, but not limited to, liver, brain, lung, pleura cavity, pericardium, peritoneum, bone, pancreas, ovary and lymphatic nodes. Particularly, the organ to which malignant tumors metastasize is liver.

In the present invention, the mechanism of inhibiting the metastasis of malignant tumors is mainly performed by blocking VEGF-C/VEGFR-3 signal transduction pathways so as to inhibit lymphangiogenesis.

The nano-gold utilized in the present invention normally has a particle size no greater than 100 nm, preferably less than 10 nm. The particle size may be determined by transmitting electronic microscope.

In the present invention, if nano-gold having a particle size less than 10 nm is utilized, its amount may be within the range of 0.01 to 5 mg/kg, preferably 0.01 to 2.0 mg/kg, and more preferably, 0.05 to 0.6 mg/kg. If nano-gold having a particle size from 10 nm to 100 nm is utilized, its amount may be within the range of 10 to 250 mg/kg, preferably 50 to 250 mg/kg, and, more preferably 150 to 250 mg/kg.

However, the specific dosage for individuals may be adjusted according to various factors, such as the severity of diseases, age, weight, general health status, gender and dietary intake, time and route of drug administration, releasing time, and whether other drugs are co-administered.

The pharmaceutical composition of the present invention may comprise any carriers known in the field of pharmaceuticals. Suitable carriers can be organic or inorganic carriers, including, for example, water, vegetable oils, polypropylene glycols, fatty acid glycerides, glycerine, soybean lecithin, carbohydrates (e.g., lactose or starch), magnesium stearate, talcum powder, and celluloses.

The pharmaceutical composition of the present invention may comprise any additives known in the field of pharmaceuticals, such as preservatives, stabilizers, excipients, emulsifiers, surfactants, buffers, color-developing agents, fragrances, and fillers.

The pharmaceutical composition of the present invention may be produced as any forms known in the field of pharmaceuticals, such as powders, particles, tablets (e.g., direct-tabletted tablets, film-coated tablets and enteric-coated tablets), capsules, liquids, syrups, and emulsions.

There are no specific limitations to the manufacture of the nano-gold utilized in the present invention. The nano-gold utilized in the present invention may be obtained from any known chemical, physical, or biological methods. However, it is preferred that the nano-gold utilized in the present invention is obtained from a physical method, because the nano-gold thus obtained is known to have lower toxicity.

As an example, the nano-gold utilized in the present invention may be produced by a process comprising the following steps:
(a) processing raw gold by, for example, cutting or grinding to form a desired target form,
(b) subjecting the gold target to an electrically gasified method in vacuum, in order to gasify the gold target to generate a gold stream,
(c) upon the gold stream reaching the top of a substrate with heat, condensing the gold stream in the presence of an inert gas, and then controlling the time for vapor deposition and the current strength in order to control the size of the gold crystals thus obtained,
(d) collecting the resulting gold crystals with a cooling trap, and
(e) centrifuging the gold crystals obtained in step (d), in order to produce nano-gold having a smaller and more homogenous particle size.

The inventors already conducted a test on the toxicity of nano-gold utilized in the present invention. It was found that the administration of the nano-gold, which is obtained by a physical method and has a particle size no greater than 10 nm, to either large or small mice, even in a dosage up to five hundred times the lower limit of recommended dosage suggested in the above, did not result in any adverse side effects no matter in genetic toxicity test or acute toxicity test.

The following examples will further demonstrate the effectiveness of nano-gold in inhibiting metastasis of malignant tumors and growth of leukemic cells.

### EXAMPLES

### Example 1

### Manufacture and Analysis of Nano-Gold

Nano-gold to be utilized in the subsequent examples was produced according the following steps:
(a) processing raw gold by, for example, cutting or grinding to form a desired target form,
(b) subjecting the gold target to an electrically gasified method in vacuum (below 10⁻⁹ torrs), in order to gasify the gold target to generate a gold stream,
(c) upon the gold stream reaching the top of a substrate with heat, condensing the gold stream in the presence of an inert gas, and then controlling the time for vapor deposition at the frequency of one shot per 30 seconds and the current strength at 126 ampere in order to control the size of the gold crystals thus obtained,
(d) collecting the resulting gold crystals having a particle size within the range of 0.1 to 80 nm with a cooling trap, and
(e) centrifuging the gold crystals obtained in step (d) at a centrifugation speed of 25,000 rpm or 40,000 rpm, so as to produce nano-gold having a particle size no greater than 10 nm.

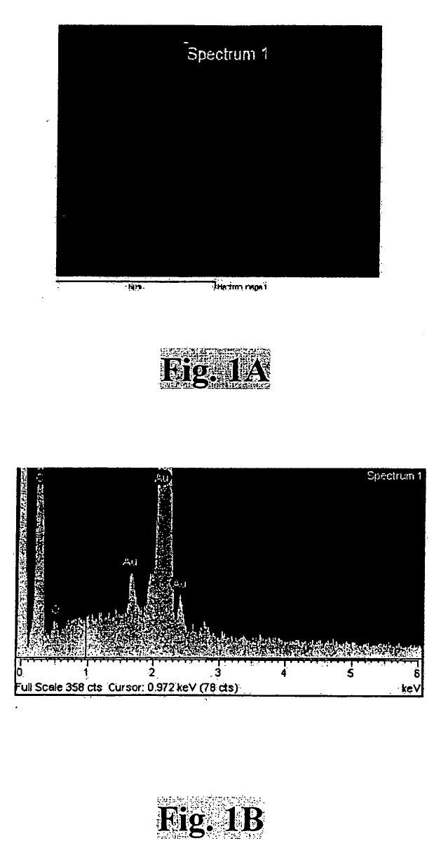

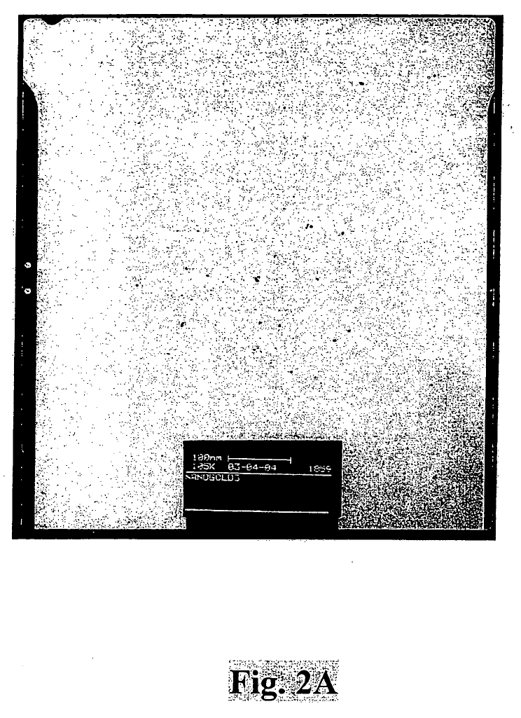

The nano-gold crystals obtained before centrifugation (namely, the nano-gold crystals obtained in step (d)) is referred to as “NG-gp.” As recited in the above, it had a particle size within the range of 0.1 to 80 nm. The particle size was determined by an energy dispersive X-ray (EDX) method. The results were shown in FIGS. 1A and 1B.

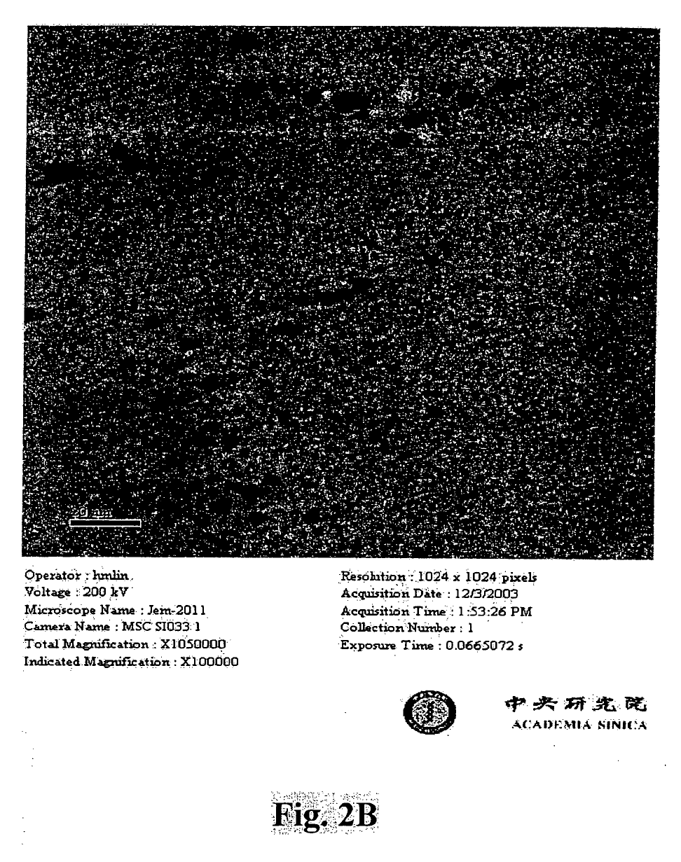

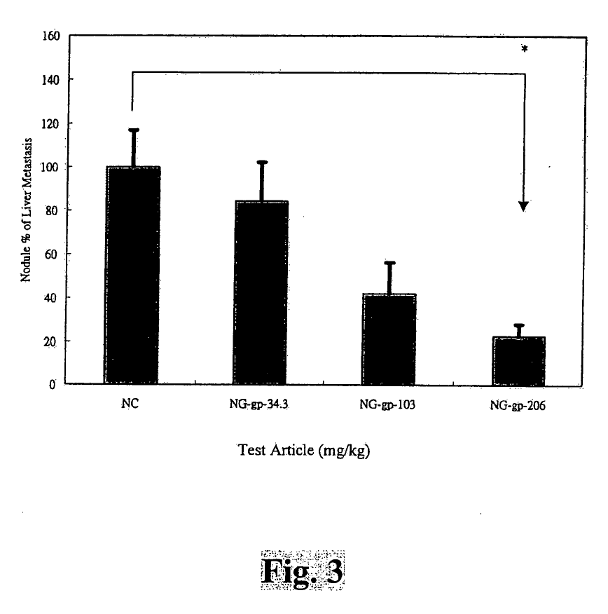

In step (e), the nano-gold comprised in the upper-layered portion obtained after centrifugation at a centrifugation speed of 25,000 rpm for one hour is referred to as “NG-25s.” The particle size of the nano-gold was determined by transmitting electronic microscope (TEM). As shown in FIG. 2A, the nano-gold had a particle size of 2.84+1.63 nm. The nano-gold comprised in the upper-layered portion obtained after centrifugation at a centrifugation speed of 40,000 rpm for one hour is referred to as “NG-40s.” The particle size of the nano-gold was determined by TEM. As shown in FIG. 2B, the nano-gold had a particle size of 2.84±1.63 nm.

### Example 2

### Evaluation of the Effectiveness of NG-gp in Inhibiting the Metastasis of Colorectal Adenocarcinoma to Liver

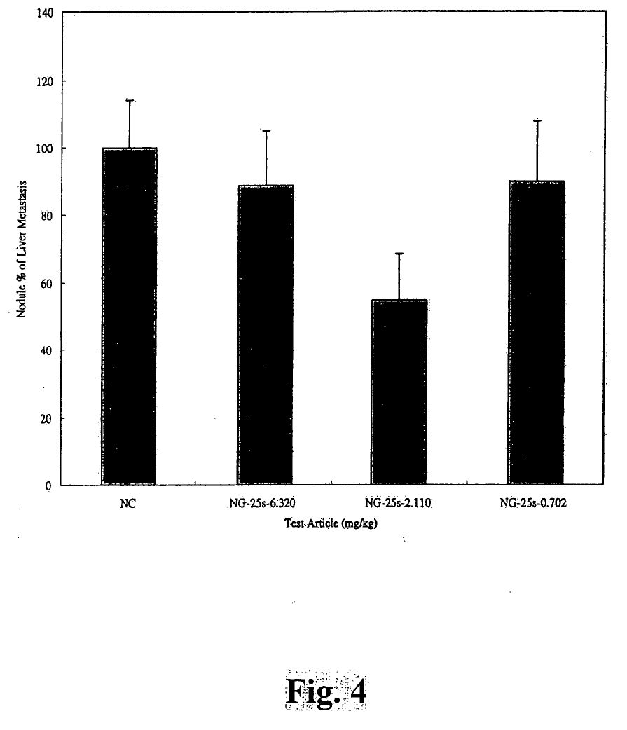

The following dosages were used to evaluate the effectiveness of a pharmaceutical composition containing NG-gp in inhibiting the metastasis of colorectal adenocarcinoma to liver:
(1) NG-gp-34.3 mg/kg (labeled as NG-gp-34.3 in FIG. 3 and classified as low dosage);
(2) NG-gp-103 mg/kg (labeled as NG-gp-103 in FIG. 3 and classified as medium dosage); and
(3) NG-gp-206 mg/kg (labeled as NG-gp-206 in FIG. 3 and classified as high dosage).
   A control group labeled as NC is also listed in FIG. 3 for comparison.

Prior to the evaluation, 6 weeks old BALB/c male mice were obtained from National Laboratory Animal Center. Each cage contained 4 mice and there were 8 mice in each group. Room temperature was set at 22±2° C., the cage was lit for 12 hours, followed by 12 hours of darkness, and each mouse was fed without any restriction. At the same time, the CT-26 colorectal adenocarcinoma cell line of mice was cultured in IMDM culture medium containing 10% cow serum at a 37° C.-5% CO₂ incubator. The CT-26 colorectal adenocarcinoma cell line of small mice was prepared for intra-splenic implantation when the mice were 8 weeks old (each mouse was implanted with 2×10⁴ colorectal adenocarcinoma cells). After weighing each mouse, an appropriate amount of anesthetic agent, pentobarbital was given according to the weight (at 10 μl/g, 6.5 mg/ml). The adjusted concentration of CT-26 colorectal adenocarcinoma cells in 100 μl was injected into the spleen of the mouse and the incision was anastomosed by staples. The mouse was returned to the cage after it recovered from the anesthetics. The mice were dissected fourteen days after the implantation of the cancer cells and the extent of metastasis of cancer cells and damage to the organs were observed. The extent of metastasis of cancer cells to the liver was compared between the groups.

The pharmaceutical compositions comprising three different amounts of NG-gp and the control group were given to the tested mice. The result was shown in FIG. 3. According to FIG. 3, NG-gp (labeled as NG-gp-206 in FIG. 3) at a high dosage was effective in inhibiting the metastasis of colorectal adenocarcinoma to the liver. (The symbol “*” in FIG. 3 indicates that the difference between the high dosage group and the control group is statistically significant with p value<0.05)

### Example 3

### Evaluation of the Effectiveness of NG-25s in Inhibiting the Metastasis of Colorectal Adenocarcinoma to Liver

The following dosages were used in the evaluation of the effectiveness of a pharmaceutical composition containing NG-25s in inhibiting the metastasis of colorectal adenocarcinoma to liver:
(1) NG-25s-6.32 mg/kg, labeled as NG-25s-6.32 in FIG. 4;
(2) NG-25s-2.11 mg/kg, labeled as NG-25s-2.11 in FIG. 4; and
(3) NG-25s-0.703 mg/kg, labeled as NG-25s-0.703 in FIG. 4.

The mice were prepared in accordance with the descriptions set forth in Example 2. After the intra-splenic implantation of a CT-26 colorectal adenocarcinoma cell line into the mice, the effectiveness of the pharmaceutical compositions containing the abovementioned NG-25s dosages in inhibiting the metastasis of colorectal adenocarcinoma to the liver was evaluated. The results were shown in FIG. 4, which indicate that NG-25 at 2.11 mg/kg dosage was effective in inhibiting the metastasis of rectal cancer cells to liver. (The symbol “*” in FIG. 4 indicates that the difference between the 2.11 mg/kg dosage group and the control group is statistically significant with p value<0.05)

### Example 4

### Evaluation of the Effectiveness of NG-40s in Inhibiting the Metastasis of Colorectal Adenocarcinoma to Liver

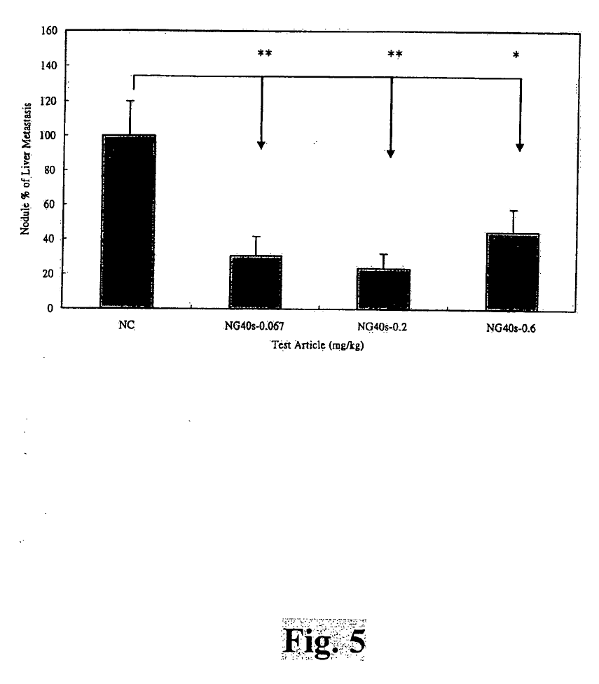

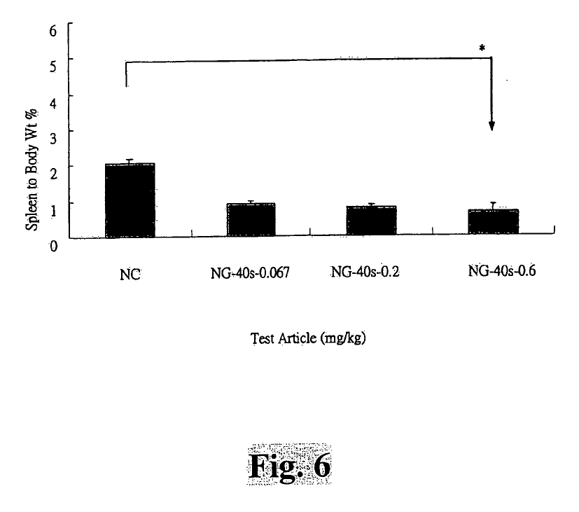

The following dosages were used to evaluate the effectiveness of a pharmaceutical composition containing NG-40s in inhibiting the metastasis of colorectal adenocarcinoma to liver:
(1) NG-40s-0.0672 mg/kg, labeled as NG-40s-0.067 in FIGS. 5, 6 and 7;
(2) NG-40s-0.2 mg/kg, labeled as NG-40s-0.2 in FIG. 5, 6 and 7; and
(3) NG-40s-0.6 mg/kg, labeled as NG-40s-0.6 in FIG. 5, 6, and 7.

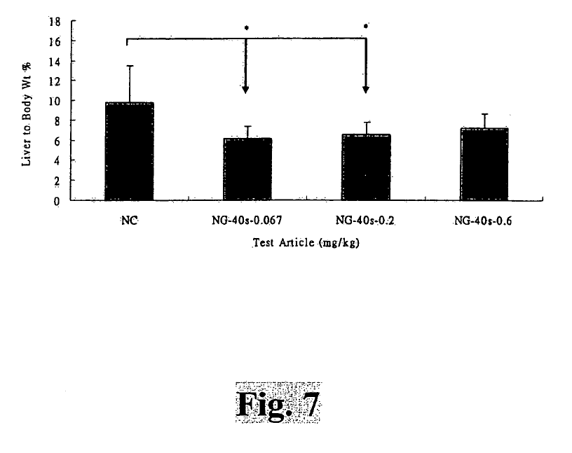

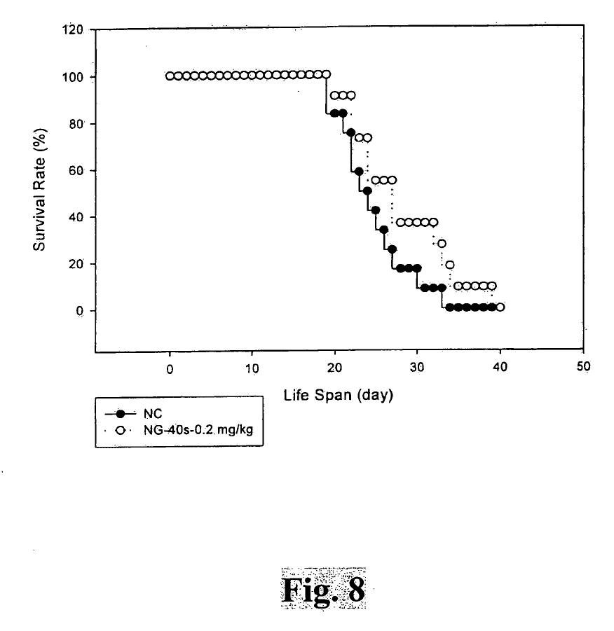

The mice were prepared in accordance with the descriptions set forth in Example 2. After the intra-splenic implantation of a CT-26 colorectal adenocarcinoma cell line into the mice, the effectiveness of the pharmaceutical compositions containing the abovementioned NG-40s dosages in inhibiting the metastasis of colorectal adenocarcinoma to the liver was evaluated. As shown in FIGS. 5 and 7, NG-40s at 0.067 mg/kg dosage was quite effective in inhibiting hepatic enlargement caused by the metastasis of colorectal adenocarcinoma to liver (The symbol “*” in FIG. 5 indicates that the difference between the 0.6 mg/kg dosage group and the control group is statistically significant with p value<0.05, and the symbol “**” indicates that the differences between 0.2 mg/kg and 0.067 mg/kg dosage groups and the control group are statistically significant with p value<0.01. The symbol “*” in FIG. 7 indicates that the difference between 0.2 mg/kg and 0.067 mg/kg dosage groups and the control group is statistically significant with p value<0.05). In addition, FIG. 6 indicates that NG-40s-0.6 was effective in inhibiting the growth of intra-splenic implanted CT-26 colorectal adenocarcinoma cell line. (The symbol “*” in FIG. 6 indicates that the difference between 0.6 mg/kg dosage group and the control group is statistically significant with p value<0.05).

### Example 5

### Evaluation of the Effectiveness of NG-40s in Prolonging the Lifespan of Mice with Implanted Colorectal Adenocarcinoma Cells

The effectiveness of a pharmaceutical composition containing 0.2 mg/kg of NG-40s in prolonging the lifespan of mice with implanted colorectal adenocarcinoma cells was evaluated.

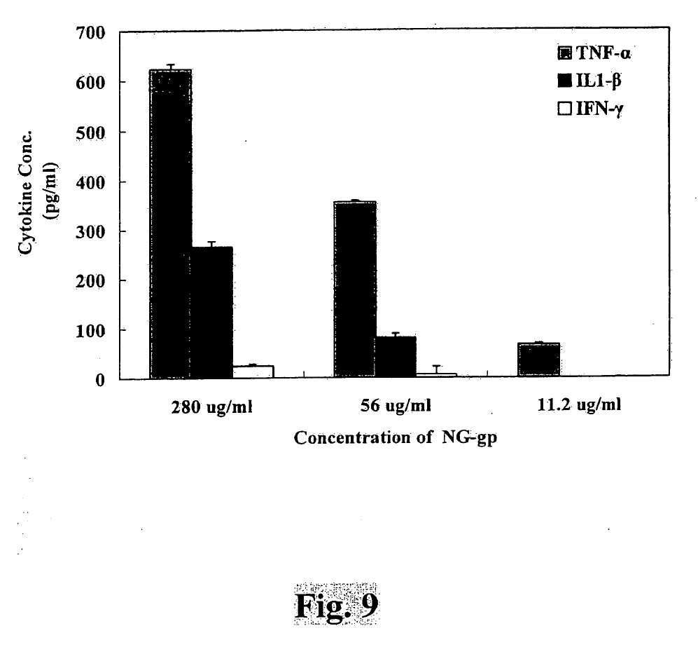

The mice were prepared in accordance with the descriptions set forth in Example 2. After the intra-splenic implantation of CT-26 colorectal adenocarcinoma cell line into the mice, the abovementioned pharmaceutical composition was continuously administered to the mice until their death. The effectiveness of the abovementioned pharmaceutical composition in prolonging the lifespan of mice with implanted colorectal adenocarcinoma cells was evaluated. The results of this experiment were shown in FIG. 8. The average lifespan was 25.3 days for the control group (NC) and 28.4 days for the treated group (NG-40s, 0.2 mg/kg). In other words, 0.2 mg/kg of NG-40 can prolong the lifespan of the mice implanted with colorectal adenocarcinoma cells by 12.3%.

### Example 6

### Evaluation of the Effectiveness of a Pharmaceutical Composition Containing Nano-Gold in Inhibiting the in vitro Growth of Leukemic Cells

1. Separation and Culture of Human Peripheral Blood Mononuclear Cells (hPBMC) and the Preparation of Conditioned Culture Medium.

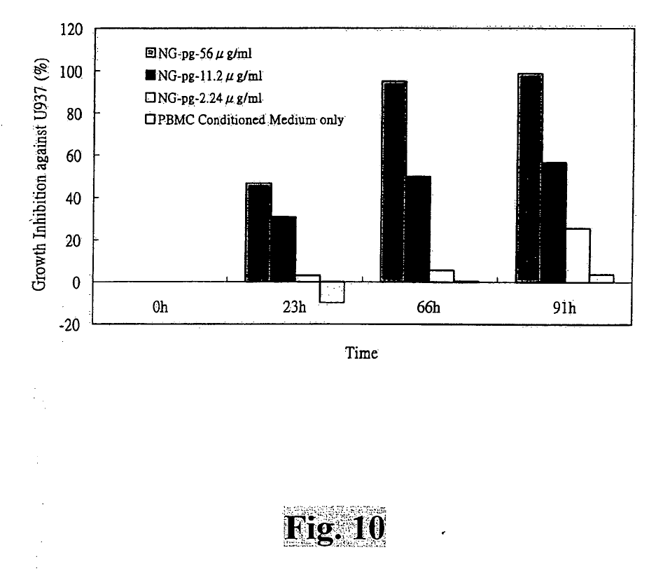

Blood cell concentrate and a Ficoll-Paque PLUS solution were added to a 50-ml centrifuge tube in 1:1 ratio and the tube was centrifuged at a centrifugation speed of 3,000 rpm for 30 minutes. The PBMC layer was carefully extracted and transferred to a new 50-ml centrifuge tube and the PBMC cells were washed three times with a complete culture medium. The cell suspension liquid was transferred to another 50-ml centrifuge tube and centrifuged at a centrifugation speed of 1,000 rpm for 5 minutes. After removing the supernatant, another appropriate culture medium was used to suspend the blood cells and the cell numbers were counted. The cell numbers were adjusted to a concentration of 1×10⁶/ml. NG-pg of various concentrations (11.2 μg/ml, 56 μg/ml, and 280 μg/ml) and PBMC were added to a micro-pore culture medium and the medium was incubated in a 37° C.-5%CO₂ incubator for 1 day. Enzymatic immunization analysis was used to analyze the concentrations of various cytokines, including tumor necrosis factor-α (TNF-α), interleukin 1-β (IL1-β) and interferon-γ(IFN-γ), as shown in FIG. 9.

2. Test of the Effectiveness of a Conditioned Culture Medium in which the Human Peripheral Blood Mononuclear Cells (hPBMC) Comprised therein were Stimulated by a Bharmaceutical Composition Containing Nano-Gold in Inhibiting the Cell Growth of a U937 Cell Line

First, a U937 cell line was recovered from a T-80 culture tube and implanted in a micro-pore culture medium. Then, 20% (v/v) of the conditioned culture mediums aforementioned were added. The micro-pore culture medium was incubated in a 37° C.-5%CO₂ incubator for 4 days. A blood cell counter was used to count the total number of cells and the number of live cells. The growth-inhibiting rate was calculated by the formula below:
Growth inhibition rate(%)=100−100×(the number of live cells in a micro-pore culture medium comprising the conditioned culture medium stimulated by the tested pharmaceutical composition/the number of live cells in a micro-pore culture medium without the conditioned culture medium)

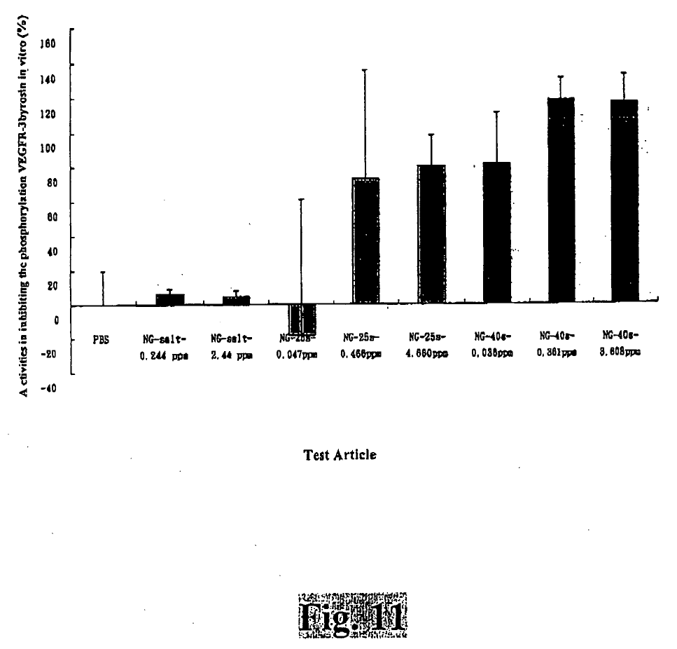

The results indicated that the conditioned culture medium, which was prepared by utilizing NG-gp to stimulate PBMC comprised therein, had a strong inhibition in the growth of a U937 cell line, as shown in FIG. 10. The effect of cell growth inhibition was positively correlated to the concentration of the various cytokines, as shown in FIGS. 9 and 10.

### Example 7

### Evaluation of the Effectiveness of Nano-Gold in Inhibiting Lymphangiogenesis

Evaluation of in vitro Activity of VEGFR-3 Tyrosine Kinase

A glutathione-S-transferase (GST) fused VEGFR-3 intracellular domain construct was implanted into a BL-21 strained E. coli expression. The E. coli was cultured until its OD₆₀₀ light absorption value reached from 0.6 to 0.8. 0.2 mM of isopropyl b-D-thiogalactoside (IPTG) was used for protein induction for 2 to 4 hours. The bacteria solution was then collected and centrifuged at a centrifugation speed of 8,000 rpm for 15 minutes, followed by suspension in cold, phosphate-buffered saline. The bacteria was broken down by using a sonicator and centrifuged at a centrifugation speed of 12,000 rpm for 20 minutes. The supernatant (i.e., total protein) was collected and purified by using a GST-4B column. The GST fusion protein containing the VEGFR-3 tyrosine kinase linase domain was eluted from the column using a reduced glutathione.

The results were shown in FIG. 11. NG-25s and NG-40s were both proven to have the activity of inhibiting VEGFR-3 tyrosine phosphorylation. However, NG-40 was most effective in inhibiting the activity of VEGFR-3 tyrosine phosphorylation and the inhibition was dosage dependent. The activity of the nano-gold obtained from a chemical method in the form of a salt was lower in this regard.

In view of the above experimental results, it can be concluded that nano-gold can effectively block the VEGF-C/VEGFR-3 signal transduction pathways. Therefore, nano-gold can inhibit lymphangiogenesis and metastasis of malignant tumors.

1. A method for inhibiting metastasis of malignant tumors, which comprise administering a pharmaceutically effective amount of nano-gold.

2. A method for inhibiting growth of leukemic cells, which comprise administering a pharmaceutically effective amount of nano-gold.

3. The method according to claim 1, wherein the nano-gold has a particle size no less than 100 nm.

4. The method according to claim 3, wherein the nano-gold has a particle size no greater than 10 nm, and the pharmaceutically effective amount is between 0.01 and 5 mg/kg.

5. The method according to claim 3, wherein the nano-gold has a particle size from 10 to 100 nm, and the pharmaceutically effective amount is between 10 and 250 mg/kg.

6. The method according to claim 1, wherein the inhibition of metastasis of malignant tumors is performed through inhibiting lymphangiogenesis.

7. The method according to claim 6, wherein the inhibition of lymphangiogenesis is performed through blocking vascular endothelial cell growth factors C (VEGF C)/VEGFR-3 signal transduction pathways.

8. The method according to claim 1, wherein the malignant tumors is colorectal adenocarcinoma.

9. The method according to claim 9, wherein the colorectal adenocarcinoma is metastasized to liver.

10. A pharmaceutical composition for inhibiting metastasis of malignant tumors comprising a pharmaceutically effective amount of nano-gold.

11. A pharmaceutical composition for inhibiting growth of leukemic cells comprising a pharmaceutically effective amount of nano-gold.

12. The pharmaceutical composition according to claim 10, wherein the nano-gold has a particle size no greater than 100 nm.

13. The pharmaceutical composition according to claim 12, wherein the nano-gold has a particle size no less than 10 nm, and the pharmaceutically effective amount is between 0.01 and 5 mg/kg.

14. The pharmaceutical composition according to claim 12, wherein the nano-gold has a particle size from 10 to 100 nm, and the pharmaceutically effective amount is between 10 and 250 mg/kg.

15. The pharmaceutical composition according to claim 10, wherein the inhibition of metastasis of malignant tumors is performed through inhibiting lymphangiogenesis.

16. The pharmaceutical composition according to claim 15, wherein the inhibition of lymphangiogenesis is performed through blocking vascular endothelial cell growth factors C (VEGF C)/VEGFR-3 signal transduction pathways.

17. The pharmaceutical composition according to claim 10, wherein the malignant tumors is colorectal adenocarcinoma.

18. The pharmaceutical composition according to claim 17, wherein the colorectal adenocarcinoma is metastasized to liver.
